(19) United States
(12) Patent Application Publication
(10) Pub. No.: US 2005/0272167 A1
(43) Pub. Date: Dec. 8, 2005

(54) Methods and systems for detecting hydrogen

(75) Inventor: Jean M. Andino, Gainesville, FL (US)

Correspondence Address: EDWARDS & ANGELL, LLP, P.O. BOX 55874, BOSTON, MA, 02205, US

(73) Assignee: University of Florida, Gainesville, FL (US)

(21) Appl. No.: 11/132,808

(22) Filed: May 18, 2005

Related U.S. Application Data
(60) Provisional application No. 60/572,712, filed on May 19, 2004.

(51) Int. Cl. G01N 33/00

(52) U.S. Cl. 436/144; 422/83

(57) ABSTRACT

Methods and systems are provided for the detection and quantification of hydrogen. Preferred methods and systems enable rapid, accurate hydrogen detection that include exposing a gaseous material to activating radiation, and measuring a change in humidity of the gaseous material following the radiation exposure to an amount of hydrogen in the gaseous material.

This application claims the benefit of U.S. provisional application No. 60/572,712, filed May 19, 2004, incorporated herein by reference in its entirety.

### 1. FIELD OF THE INVENTION

The invention relates generally to methods and systems for the detection and quantification of hydrogen. More particularly, methods and systems are provided for rapid, accurate hydrogen detection that include exposing a gaseous material to activating radiation, and measuring a change in humidity of the gaseous material following the radiation exposure to an amount of hydrogen in the gaseous material.

### 2. BACKGROUND

Alternative energy sources are rapidly becoming a global necessity as traditional sources of energy become insufficient to cheaply and cleanly meet the energy requirements of modern society. Fuel cells have become favored as such a potential alternative energy source. The projected applications for fuel cells include powering small electronic devices, powering automobiles, heating homes, and serving as urban power plants. The basic principle of a fuel cell is to convert a supply of hydrogen and oxygen into water and, thereby, produce useful heat and electricity.

The oxygen supply for a fuel cell may simply draw upon ambient air. Supplying hydrogen however can be more difficult task. For example, when powering an automobile, transients such as stopping and accelerating provide intervals in which engine output varies. In the prior art, however, hydrogen concentration has been maintained at a maximum level because of an inability to modify hydrogen concentration at appropriate intervals. It can be seen that the requirement of providing excess hydrogen is inefficient.

For another example, as the lightest of molecules, hydrogen was used to provide lift for the Hindenberg air ship. Despite the accepted conclusion that the use of hydrogen did not cause the disastrous crash of the Hindenberg air ship, the general public has a mistrust of flammable hydrogen in public use. Further, hydrogen is highly flammable and leaks do pose real hazards. In view of this, use of a quick, reliable and accurate detection means where hydrogen leaks may occur would allay safety concerns as well as allow remediation of the leak.

Prior approaches to detecting hydrogen have been unable to meet the requirements above. Typical approaches have employed gas chromatography and methods using metal membranes. See, for instance, U.S. Pat. Nos. 5,668,301 and 5,670,115 and U.S. Patent Publication 20030153088. These approaches often have proved costly and too slow to be effective for many applications.

Other complex hydrogen detection systems have been reported directed at particular applications such as for use in nuclear fusion reactors. However, such applications of hydrogen sensing are not practically adapted to a variety of current and foreseeable uses. For instance, U.S. Pat. No. 5,932,797 reports use of a vacuum system for hydrogen leak detection.

It thus would be desirable to have new systems and methods for detecting hydrogen that can readily be adapted to the ever expanding practical applications for using hydrogen. It would be particularly desirable to have new systems that would be scalable for various applications, yet provide fast, reliable and accurate results.

### SUMMARY OF THE INVENTION

We now provide new methods and systems for quantifying hydrogen levels in air such as for discrete collected air samples or air flow through a conduit. Methods and systems of the invention can quickly, accurately and reliably determine hydrogen levels in air.

Preferred methods of the invention include i) exposing a gaseous material to activating radiation such as ultraviolet radiation, ii) measuring a change in humidity resulting from the exposure step and iii) correlating the change to a percentage of hydrogen.

In a preferred system, a device receives an air sample to be measured. A radiation source (particularly, an ultraviolet light source) photolyzes an air sample containing hydrogen (hydrogen/air mixture sample) to generate water vapor that can be read by a hygrometer. The resulting change in humidity is correlated to the percentage of hydrogen.

In another preferred system of the invention, in place of hygrometer, an infrared detection system is employed. In one such system, a housing defines a reference portion for retaining ambient air and a sample portion for retaining an air sample to be measured. An infrared source provides a beam into the reference and sample portions such that differential heating occurs therebetween. A diaphragm between the reference portion and the sample portion moves in proportion to the differential heating, to thereby, provide data to a signal processor for determining a level of hydrogen concentration in the sample portion.

Indeed, methods and systems of the invention provide effective detection of hydrogen without the long delay and complex components of the prior art.

As mentioned above, a variety of applications may benefit from the systems and methods of the invention, including fuel cells, automobiles and any location in which hydrogen is supplied. For instance, for fuel cell applications, by rapid monitoring of hydrogen flow within relevant intervals through use of a system of the invention, the hydrogen supply and, thereby, the power output may be varied appropriately to increase fuel cell efficiency.

Other aspects of the invention are disclosed infra.

### BRIEF DESCRIPTION OF THE DRAWINGS

FIG. 1 shows schematically a hydrogen-detecting device of the invention;

FIG. 2 shows schematically another exemplary hydrogen detecting device of the invention;

FIG. 3 depicts schematically a top cross-sectional view of the sample portion of the device of FIG. 2;

FIG. 4 depicts schematically a front cross-sectional view of the sample portion of the device of FIG. 2; and

FIG. 5 shows results of Example 1 which follows.

### DETAILED DESCRIPTION OF THE INVENTION

As discussed above, systems and methods of the invention will be particularly useful for detecting hydrogen leaks and providing feedback in hydrogen delivery systems. Existing systems also can be effectively retrofitted with a device in accordance with the invention.

Significantly, the hydrogen detecting methods and devices of the invention can be scaled for use with very small systems as well as very large systems. For instance, a hydrogen detecting system of the invention can be portable for an individual to carry or integrated into common small electronic devices.

Additionally, the detection of hydrogen can be accomplished in near real-time so that the hydrogen-detecting device of the invention can be utilized for various applications. For example, the hydrogen-detecting device can be utilized as a feedback mechanism to monitor the delivery of hydrogen to a fuel cell.

In generally preferred systems of the invention a device or container receives an air sample to be measured. That contained sample is then exposed to activating radiation such as ultraviolet light that can result in the generation of water vapor which can be conveniently detected by a hygrometer or other detection system. The resulting change in humidity is correlated to a percentage of hydrogen in the air sample. Radiation is considered activating for a gaseous sample if exposure of a sample with the radiation results in generation of water vapor, such as through photolysis of hydrogen and other material(s) present in the gaseous sample.

As discussed above, rather than a hygrometer, an infrared detection system may be employed, which may suitably comprise a housing that defines a reference portion for retaining reference air, and a sample portion for retaining an air sample to be measured. The gaseous sample is exposed to a source of activating radiation, such as an ultraviolet light source which can photolyze material(s) within the air sample. An infrared source provides a beam into the reference and sample portions such that differential heating occurs therebetween because of the photolysis. A diaphragm between the reference portion and the sample portion moves in proportion to the differential heating, to thereby, provide data to a signal processor for determining a level of hydrogen concentration in the air sample.

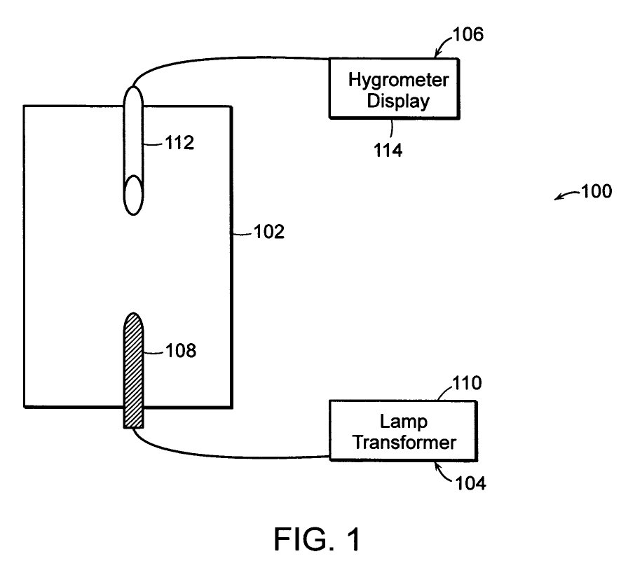

Referring now to the drawings, in FIG. 1 a preferred hydrogen-detecting device of the invention is schematically shown and referred to generally by the reference numeral 100. The device 100 captures a sample to be measured in a container 102. An ultraviolet light source 104 photolyzes hydrogen and other material(s) within the sample to generate water vapor. A hygrometer 106 measures the resulting change in humidity within the container 102 that correlates with the percentage of hydrogen.

The container 102 can be of a variety of a variety of configurations and materials of constructions and suitably will be a plastic or metal construction and adapted to couple with the light source 104 and hygrometer 106 thereto. For instance, the container 102 can be a rigid plastic housing with mounting holes for threadably engaging the light source 104 and hygrometer 106. Preferably, the rigid plastic housing has tubing coupled thereto for receiving samples. Alternatively, the container 102 may be a flexible material as exemplified in the system of Example 1, which follows. Gaseous samples for analysis can be introduced into a container 102 by a variety of methods, including e.g. by a rotameter.

The ultraviolet light source 104 suitably is a relatively short wavelength radiation source for photolyzing hydrogen and other material(s) of a gaseous sample, e.g., ultraviolet radiation having a wavelength of about 254 nm or less. Preferably, only a lamp 108 of the light source 104 is located within the container 102. Thus, the transformer 110 for powering the lamp 108 can be easily accessible by the user.

The hygrometer 106 for measuring humidity is a well known instrument. Similar to the light source 104, only the hygrometer probe 112 needs to be located within the container 102. The hygrometer display 114 for presenting the readings to the user does not need to be within the container 102. In another embodiment, an Fourier Transform Infrared (“FTIR”) spectrometer is used to quantify the level of water vapor.

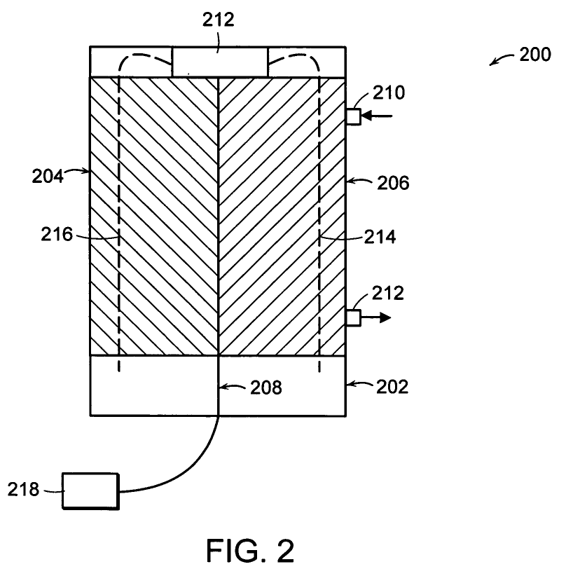

Referring now to FIG. 2, an alternatively configured device 200 of the invention is depicted. In the device 200, a modified infrared system is used for the detection of water vapor. The device 200 is based on the use of a non-dispersive infrared (NDIR) system that has been used in the past for carbon monoxide (CO) detection. The device 200 uses an NDIR system adapted with an ultraviolet photolysis system to quantify the presence of hydrogen.

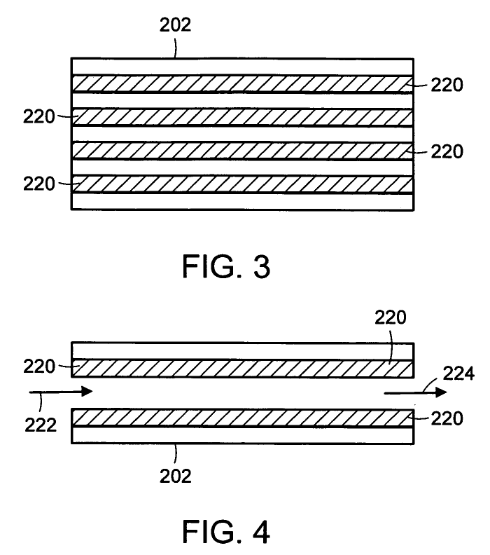

The device 200 has a sensing compartment 202 partitioned into a reference portion 204 and a sample portion 206. A diaphragm 208 extends through and defines sensing compartment 202 as generally shown in FIG. 2, but does not extend into the areas of reference portion 204 and sample portion 206. The reference portion 204 contains background or ambient gas. The gas to be measured flows through the sample portion 206 by an inlet 210 and an outlet 212 formed in the sensing compartment 202. An infrared source 212 provides a split infrared beam so that one beam 214 flows through the sample portion 206 of the sensing compartment 202 and the other beam 216 flows through the reference portion 204. As shown in FIGS. 3 and 4, an ultraviolet light source photolyzes the hydrogen and other material(s) within the sample portion 206.

Referring now to FIGS. 3 and 4, top and front cross-sectional views of the sample portion 206 of the device 200 of FIG. 2 are shown, respectively. In FIG. 4, arrows 222, 224 designate the flow direction of the gas to be measured. The sample portion 206 of the sensing compartment 202 has a plurality of light rods as an ultraviolet light source 220. The light rods of the ultraviolet source 220 could be selected from ultraviolet lamps and the like for large scale applications. For a small scale applications, GaN rods or other such light emitting diodes and rods that provide ultraviolet light can be employed.

Referring again to FIGS. 2, 3 and 4, the ultraviolet light source 220 photolyzes the hydrogen and other material(s) present within the gas (air) sample passing through the sample portion 206. As the beam 214 passes through the sample portion 206, the beam 214 interacts with the water vapor and, in turn, the infrared signal decreases in the sample portion 206. The decrease in the infrared signal in the sample portion 206 as compared to the reference portion 204 causes a differential heating. As a result, the diaphragm 208 moves proportionally. The diaphragm 208 is coupled to a signal processing system 218 for correlating diaphragm movement to the concentration of hydrogen in the sample portion 206. The signal processing means 218 can be, without limitation, a computer, a special purpose microprocessor and like electronic circuitry for accomplishing the required function as would be known to those of ordinary skill in the art. For many applications, it may be preferred to vary the length of the sensing compartment 202 to attain the desired photolysis time for the device 200. In practice, the device 200 would need to be fully calibrated by empirical methods or otherwise.

Systems and methods of the invention will be particularly useful for hydrogen fuel cell systems. More particularly, the device 200 can be incorporated directly on-line with a hydrogen fuel cell system to provide feedback for monitoring the hydrogen delivery. Fast feedback allows for accurate control of the hydrogen delivery. Accurate hydrogen delivery will enhance the performance of fuel cell based systems by optimizing fuel delivery to meet demand. In short, the device 200 would make hydrogen fuel cell systems more efficient.

Further, the systems and methods of the invention can be incorporated to measure hydrogen leaks in such applications as varied as NASA, the fuel cell industry, and analytical laboratories that utilize hydrogen in gas-chromatography systems. The resulting fast and reliable leak detection would help to minimize risks posed by hydrogen leaks and allay public concern.

All documents mentioned herein are incorporated herein by reference in their entirety. The following non-limiting example is illustrative of the invention.

### EXAMPLE 1

A 15-liter Teflon bag was filled repeatedly with air/hydrogen mixtures having varying hydrogen concentrations. A hygrometer (measures relative humidity) and ultraviolet radiation source (emitting radiation having a wavelength of about 185 nm and 254 nm) were in communication with the bag samples, generally corresponding to the system depicted in FIG. 1 of the drawings. Each of the hydrogen/air mixtures within the bag was photolyzed with the UV radiation (4 minutes photolysis per sample) and responses were detected using a hygrometer. The relative humidity values measured by the hygrometer were converted to an absolute water concentration using the temperature and saturation vapor pressure of water. The change in water vapor concentration was calculated in view of water vapor present in initial air samples.

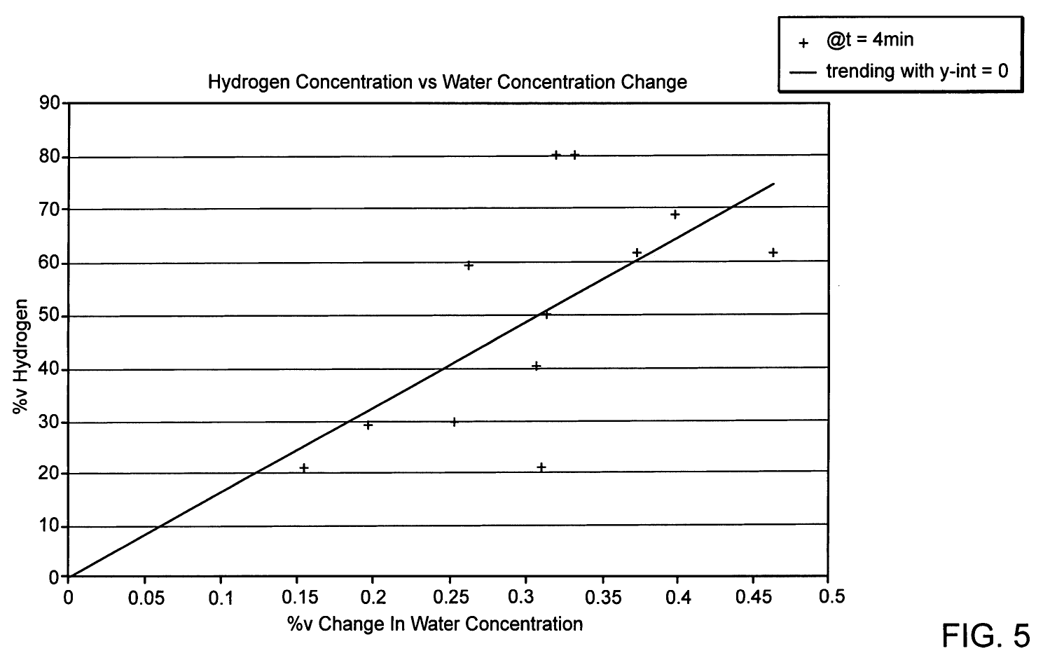

Data from these measurements as set forth in FIG. 5 of the drawings indicates that the relationship between hydrogen and change in water vapor concentration is relatively linear and shows that water vapor concentration increases with increasing hydrogen concentrations.

A sample container smaller than the 15-liter bag used for these measurements would decrease the appropriate mass transfer time (i.e., the time for the water vapor to reach the water vapor sensing element) and, thereby, the response time. Thus, a smaller sample container would permit use of a shorter photolysis time than employed for these measurements.

The invention has been described in detail with reference to particular embodiments thereof. However, it will be appreciated that those skilled in the art, upon consideration of this disclosure, may make modifications and improvements within the spirit and scope of the invention.

1. A method for detecting hydrogen, comprising:
(a) exposing a gaseous material to activating radiation; and
(b) measuring a change in humidity of the gaseous material following the radiation exposure to an amount of hydrogen in the gaseous material.

2. The method of claim 1 wherein the gaseous material is exposed to ultraviolet radiation.

3. The method of claim 1 further comprising calculating humidity of the gaseous material prior to exposing the material to radiation.

4. The method of claim 1 wherein the gaseous material is a discrete sample collected within a container.

5. The method of claim 1 wherein the gaseous material flows during exposing and/or measuring.

6. The method of claim 1 wherein the gaseous material is associated with a fuel cell.

7. A device for detecting hydrogen comprising:
(a) a container for retaining a gaseous sample;
(b) a radiation source coupled to the container;
(c) a device for measuring humidity of the sample within the container; and
(d) processing means for receiving the humidity measurement to measure hydrogen in the sample.

8. The device of claim 7 wherein the sample is a flowing supply of hydrogen for a fuel cell.

9. A device for detecting hydrogen comprising:
(a) a container for retaining a sample;
(b) a radiation source coupled to the container; and
(c) means coupled to the container for measuring hydrogen content in the sample.

10. The device of claim 9 wherein the sample is a flowing supply of hydrogen for a fuel cell.

11. The device of claim 9 wherein the means is a hygrometer coupled to a signal processor.

12. The device of claim 9 wherein the means is a FTIR spectrometer.

13. A device for detecting hydrogen comprising:
(a) a compartment having a reference portion for retaining ambient air, and a sample portion for retaining an air sample to be measured;
(b) a radiation source in communication to the sample portion; and
(c) an infrared radiation source for providing infrared radiation to the reference and sample portions.

14. The device of claim 13 further comprising a diaphragm between the reference portion and the sample portion for moving in response to the differential heating induced by the infrared radiation source.

15. The device of claim 14 wherein differential heating occurs between the reference portion and the sample portion when the sample has a different hydrogen concentration from the reference.

16. The device of claim 13 further comprising a signal processor coupled to the diaphragm for determining a level of hydrogen concentration based upon movement of the diaphragm.

17. The device of claim 13 wherein the ultraviolet light source is a plurality of light emitting diodes.

18. A device for detecting hydrogen comprising:
first means for photolyzing a gaseous material;
second means for measuring a change in humidity of the gaseous material; and
third means coupled to the second means for correlating the change to a percentage of hydrogen.

19. The device of claim 18 wherein the first means is an ultraviolet light.

20. The device of claim 18 wherein the second means is a hygrometer.

21. The device of claim 18 wherein the third means is a computer.
